(19) United States
(12) Patent Application Publication
(10) Pub. No.: US 2023/0183823 A1
(43) Pub. Date: Jun. 15, 2023

(54) METHODS TO DETECT A VIRUS IN A BIOLOGICAL SAMPLE

(71) Applicant: The Trustees of Indiana University, Bloomington, IN (US)

(72) Inventors: Hunter Matthias Gill, Coatesville, IN (US); Sarath Chandra Janga, Carmel, IN (US); Quoseena Mir, Indianapolis, IN (US)

(21) Appl. No.: 17/997,595

(22) PCT Filed: Apr. 30, 2021
(86) PCT No.: PCT/US21/30354

§ 371 Date: Oct. 31, 2022

Related U.S. Application Data
(60) Provisional application No. 63/017,987, filed on Apr. 30, 2020.

(51) Int. Cl. C12Q 1/70; C12N 7/00

(52) U.S. Cl. CPC C12Q 1/70; C12N 7/00; C12N 2770/20022

(57) ABSTRACT

Disclosed are methods to characterize at least one vims in at least one human patient by (a) extracting a viral polynucleotide from a biological sample from the at least one human patient, (b) sequencing the viral polynucleotide to generate viral polynucleotide sequence data; and, (c) characterizing the viral polynucleotide sequence data. Further aspects of the invention may include a system which enables user to quickly and accurately search and/or add to data bases that facilitate the identification and/or treatment of diseases caused by viruses.

### PRIORITY CLAIM

This application claims the benefit if U.S. provisional patent application No. 63/017,987 which was filed on Apr. 30, 2020 and is incorporated by reference in its entirety.

### GOVERNMENTAL SUPPORT

This invention was made with government support under 1940422 awarded by the National Science Foundation. The government has certain rights in the invention.

### FIELD OF THE INVENTION

Aspects of the invention relate to methods of detecting, characterizing, and treating diseases caused by viruses identified in samples retrieved from human or animal patients.

### BACKGROUND

Viruses are small infectious agents which are mostly comprised of a polynucleotide either single or double stranded RNA/DNA surrounded by a protein capsid, the capsid itself may or may not be surrounded by a envelop which may itself include proteins. Viruses can only reproduce by invading living cells and using the systems of the invaded cell or replicate the components of the next generation of virus particles. Many more viruses are thought to exist than have been identified. Many of the known viruses cause diseases in human and animals. Diseases caused by viruses include but are by no means limited to the common cold, flu, or fatal even diseases like HIV-AIDS. The sheer number of different viruses and their ability to evolve over time, make it difficult to identify and track their evolution.

Examples of pathogenic viruses that evolve in real time include human respiratory viruses (HRV). HRVs are a set of viruses that infect the upper or lower respiratory track and span several families, including rhinoviruses, orthomyxoviruses, and coronaviruses. Infection with some HRVs typically results in mild illness while others cause acute viral infection and are a major source of mortality worldwide. HRVs with high transmissibility spark local epidemics or global pandemics. Species from the coronavirus family have resulted in notable outbreaks of viral disease, including the 2002-2004 SARS-CoV-1 epidemic and 2012 MERS outbreak [4-5]. More recently, a novel coronavirus first detected in 2019 has set off a pandemic, sickening and killing millions of people.

The threat posited by human respiratory viruses is underscored by the explosive global spread of severe acute respiratory syndrome coronavirus 2 (SARS-CoV-2), the causative agent of coronavirus disease 2019 (COVID-19) In the early stage of the pandemic caused by this virus the case fatality ratio was highest for at-risk populations including older adults and individuals with comorbidities and recovered patients can be left with long-term effects due to vascular endothelial damage and neuroinvasion Widespread transmission of COVID-19 has resulted in approximately 86 million cases and 2 million deaths worldwide with nearly 21 million cases and 500,000 deaths in the United States As health agencies combat new cases, viral diagnostic testing approaches provide an effective means for monitoring the pandemic.

The ability of this virus to infect large numbers of people and to rapidly evolve make it difficult to treat and to manage its spread. Viral strains like this one can be difficult to detect and easy to transmit, leading to the emergence of pandemics. In these cases, it is important to have a method to detect the virus as fast as possible, to prevent its transmission and efficiently treat the symptomatic patients.

### BRIEF DESCRIPTION OF THE DRAWINGS

FIG. 1 shows a schematic of a protocol for rapid virus detection and screening according to an embodiment of the invention.

FIG. 2 shows a UCSC genome track with the SARS-CoV-2 genome and primers obtained from Artic-network for amplicon sequencing according to an embodiment of the invention.

FIG. 3 shows a computational pipeline for amplicon sequence processing and analysis and corresponding visualization tools for real time monitoring of the viral presence, evolution and surveillance according to an embodiment of the invention.

FIG. 4A an illustration showing the primer prediction and visualization workflow for one of more of the embodiments.

FIG. 4B an illustration showing the major steps in using either short amplicons or long amplicon sequencing approaches to identify different viruses and/or different variants of the same virus present in a sample.

FIG. 5A an illustration of a database of viral genome sequence tracks along with tracks showing the coding sequences, partitions created for primer design and designed primers at differing levels of specificity. SARS-COV2 genome is highlighted in this genome browser screenshot.

FIG. 5B Screenshot showing a small selection of primers along with various properties for detecting the presence of SARS-COV2 in clinical samples via the proposed primer design system.

FIG. 6A a graph showing the proportion of the designed PCR primers exhibiting different categories of specificity for the 150-200 nt amplicon size range for different viruses. Specificity of a primer is defined based on the extent of conservation of the genomic region being captured.

FIG. 6B a graph showing the proportion of the designed PCR primers exhibiting different categories of specificity for the 300-500 nt amplicon size range for different viruses.

FIG. 6C a graph showing the extent of genomic coverage obtained using the designed primers from different specificity categories for the 150-200 nt amplicon size ranges for different viruses.

FIG. 6D a graph showing the extent of genomic coverage obtained using the designed primers from different specificity categories for the 300-500 nt amplicon size ranges for different viruses.

FIG. 7A a gel showing the detection of amplicons from SARS-CoV-2 clinical sample using the short-range primer pairs.

FIG. 7B a gel showing the detection of amplicons from SARS-CoV-2 clinical sample using the long-range primer pairs.

FIG. 8A schematic overview of the system used to practice some embodiments of the invention.

BRIEF DESCTIPION OF THE SEQUENCES

| | | |
|---|---|---|
| SEQ ID NO. 1. | GAGCTGGTAGCAGAACTCG | Forward Primer |
| SEQ ID NO. 2. | GTAGCTTGTCACACCGTTTC | Forward Primer |
| SEQ ID NO. 3. | AACTCAAGCCTTACCGCAGA | Forward Primer |
| SEQ ID NO. 4. | ACTCAAGCCTTACCGCAGA | Forward Primer |
| SEQ ID NO. 5. | CTTGTGCTGCCGGTACTAC | Forward Primer |
| SEQ ID NO. 6. | TGCTATTGGCCTAGCTCT | Forward Primer |
| | CTACT | |
| SEQ ID NO. 7. | ACTTCCTTGGAATGTAGT | Forward Primer |
| | GCGT | |
| SEQ ID NO. 8. | ACGTGGTTGACCTACACAG | Forward Primer |
| SEQ ID NO. 9. | GATCGGCGCCGTAACTATG | Reverse Primer |
| SEQ ID NO. 10. | TTGGCCGTGACAGCTTGACA | Reverse Primer |
| SEQ ID NO. 11. | TCTGCATGAGTTTAGGCC | Reverse Primer |
| | TGA | |
| SEQ ID NO. 12. | CTGCATGAGTTTAGGCCTGA | Reverse Primer |
| SEQ ID NO. 13. | GTAGACGTACTGTGGCAGC | Reverse Primer |
| SEQ ID NO. 14. | CTAGTGTGCCCTTAGTTAGCA | Reverse Primer |
| SEQ ID NO. 15. | TGGACAGCTAGACACCTAGT | Reverse Primer |
| SEQ ID NO. 16. | CTGCATGAGTTTAGGCCTGA | Reverse Primer |

### SUMMARY

One embodiment of the invention is a method to characterize at least one virus in at least one human patient by (a) extracting a viral polynucleotide from a biological sample from the at least one human patient, (b) sequencing the viral polynucleotide to generate viral polynucleotide sequence data; and, (c) characterizing the viral polynucleotide sequence data. The viral polynucleotide sequence data generated may be targeted viral polynucleotide sequences or single molecule viral genome sequences. The step of characterizing the generated viral polynucleotide sequence data may include reconstructing a viral genome, determining evolutionary relationships and abundance of the viral specie, and/or determining a clinical risk associated with the presence of the virus in the patient. The method may be a point-of-care, real-time method to characterize the at least one virus from a plurality of different biological samples from human patients. The viral polynucleotide may be a viral RNA or DNA. The at least one virus may be at least two viruses where one virus is a coronavirus. The coronavirus may be severe acute respiratory syndrome coronavirus 2 (SARS-CoV-2). The biological sample from the at least one human patient may be stool, blood, urine, a mucus sample, a saliva sample, a sputum sample, sweat, tears, plasma and lymph fluid. Methods of the invention may also include a step of processing the viral polynucleotide to add or to remove a unique barcode identifier with the viral polynucleotide where the barcode identifier represents metadata identifying a source sample from which the biological sample was taken and the unique barcode identifier is configured to form a unique, repeatable, characteristic signature when read during the sequencing step. The sequencing step may be performed by any ultra-high-throughput sequencing technology such as Illumina/Solex, SOLiD, Roche/454, PacBio, Ion Torrent and long-read nanopore processes such as an Oxford Nanopore MinION sequencer. The step of characterizing the targeted viral polynucleotide sequence data may include the step of detecting whether one or more types of viruses are present in the biological sample and documenting their relative composition in the sample. The step of characterizing the targeted viral polynucleotide sequence data may include providing strain information about a specific virus that is present in the biological sample. The step of characterizing the targeted viral polynucleotide sequence data may include providing viral burden information about a virus that is present in the biological sample. The step of characterizing the targeted viral polynucleotide sequence data yields information on co-infection of multiple viruses in a biological sample to facilitate therapeutic decisions and combinatorial vaccine therapies. The step of characterizing the targeted viral polynucleotide sequence data may be completed upon obtaining a desired result or in real time as the sequence data is resulting from mobile or benchtop sequencers which are readily deployed at the point of care. The step where the data analysis of the resulting sequencing data can be performed either locally or in a remote server to provide information to the end user on smart phone or mobile devices to facilitate at home testing.

A first embodiment includes a method for characterizing at least one virus and/or at least one variant of a virus and/or treating a disease caused by the virus in a sample collected from a human or an animal patient, comprising: extracting at least one viral polynucleotide from a biological sample from the at least one patient, sequencing the viral polynucleotide to generate viral polynucleotide sequence data; and, characterizing the viral polynucleotide sequence data.in some embodiments that separation step is performed on two or more samples simultaneously. The isolation of viral RNA and/or DNA can be accomplished using instruments and or reagents intended for or adapted to use for this purpose. Processing multiple samples from multiple patients in parallel saves considerable time and is one preferred method for accomplishing the isolation of viral polynucleotides for further analysis.

A second embodiment of the invention includes the methods of the first embodiment wherein the sequencing step is performed to generate either, or both, targeted viral polynucleotide sequence data and/or single molecule viral genome data. These steps may include sequencing the entire or virtually the entire genome of one or more virus in a single given virus. Whole genome sequencing of one or more viruses or viral variants in a given sample, either with or without the use of primer, allows for a rapid identification of specific viruses or variants of virus and is particularly useful in a samples includes more than one virus or a still unidentified or not well known variant of a known virus. In addition to be useful for the identification of a virus whole sequence information can be used to help treat infections caused by diseases, this information can also be used to generated primers for use in the analysis of viral RNA or DNA using methods that may not require whole genome sequencing. Sequence information may be saved local or remotely or both, once collected the data can added to any accessible local or remote data base.

A third embodiment of the invention includes any of the methods of the first and/or the second embodiments, wherein the viral polynucleotide sequence data which is obtained is used to reconstruct the genome of the virus, to determine, for example the evolutionary relationships and abundance of the viral specie, and/or to determine a clinical risk associated with the presence of the virus in the patient. Such information selected from multiple individual patients may be compared and used to map the spread of a given virus or given variant of a virus within or across populations.

A fourth embodiment of the invention includes performing the steps outlined in the first through the third embodiments of isolating viral polynucleotides from one or more samples form one or more patients and determining the whole sequence or a least a part of the sequence of a virus is performed at the point of care. Point of care locations, include but are not limited to hospitals, clinics, physicians' offices, schools, workplaces, public or private facilities, essentially anywhere so equipped to lawfully collect and process biological samples from a human or an animal. Sequencing may be conducted in ‘real time’ or example that results of the sequence analysis may be available within minutes, hours, or in some cases less than 1 day of beginning the analysis.

A fifth embodiment of the invention includes and of the embodiment of the first through the fourth embodiments where the viral polynucleotide is a viral RNA or DNA.

A sixth embodiment of the invention includes any of the methods according to first through the fifth embodiments wherein in the virus is one or more viruses, in some embodiments at least one of the viruses is a coronavirus.

A seventh embodiment of the invention includes any of the methods according to sixth embodiment wherein the at least one coronavirus is severe acute respiratory syndrome coronavirus 2 (SARS-CoV-2).

An eighth embodiment of the invention includes any of the method of the first through the seventh embodiments wherein the biological sample from the at least one human patient is a nasopharyngeal sample, a mucus sample, a saliva sample, a sputum sample, a bronchial aspirate and a serum sample.

A ninth embodiment of the invention includes any of the method of first through the eighth embodiments further including the step of processing the viral polynucleotide to add or to remove a unique barcode identifier with the viral polynucleotide where the barcode identifier represents metadata identifying a source sample from which the biological sample was taken and the unique barcode identifier is configured to form a unique, repeatable, characteristic signature when read during the sequencing step. The use of identifies unique to samples collected from specific individual patients or specific pools of patients and/or unique to specific primers allows for faster processing of large number of samples.

A tenth embodiment of the invention includes any of the methods of the first though the ninth embodiments wherein one or more of the sequencing steps is a high-throughput sequencing step.

An eleventh embodiment of the invention includes of the method of first through the tenth embodiments where the sequencing step is performed by a nanopore process and the nanopore process utilizes an Oxford Nanopore MinION sequencer. Any device or reagent that can rapidly isolate viral RNA and or DNA from a biological sample and/or rapidly sequence the isolate and viral RNA and or DNA recovered from a biological sample from a human or an animal can be used to practice the invention.

A twelfth embodiment includes any of the methods of the first through the eleventh embodiments wherein the step of characterizing the targeted viral polynucleotide sequence data includes detecting whether a virus is present in the biological sample.

A thirteenth embodiment incudes any of the methods of first through the eleventh embodiments wherein the step of characterizing the targeted viral polynucleotide sequence data includes providing strain information about a virus that is present in the biological sample.

A fourteenth embodiment incudes any of the methods of first through the eleventh embodiments wherein the step of characterizing the targeted viral polynucleotide sequence data includes providing viral burden information about a virus that is present in the biological sample

A fifteenth embodiment incudes any of the methods of the first through the eleventh embodiments where the step of characterizing the targeted viral polynucleotide sequence data is completed upon obtaining a desired result.

A sixteenth embodiment includes any of the methods of the first through the fifteenth embodiments wherein the sequencer generating the targeted viral polynucleotide sequence data is stopped, upon determining the presence of the virus in a sample in real time.

A seventeenth embodiment includes any of the methods of the first through the sixteenth embodiments wherein the sequenced viral genomes from an individual patient sample provide the identity of the strain, species and abundance of the viruses enabling real time understanding of the evolution of the virus.

An eighteenth embodiment includes any of the methods of the first through the sixteenth embodiments wherein the sequencing data yields information on co-infection of multiple viruses in a patient sample to facilitate therapeutic decisions and combinatorial vaccine therapies.

A nineteenth embodiment includes any of the methods of the first through the eighteenth embodiments wherein the data analysis of the resulting sequencing data can be performed in a remote server to provide information to the end user on smart phone or mobile devices.

A twentieth embodiment includes any of the methods for where the experimental protocol for isolating the virus can involve the use of specific primers targeting one or more virus of interest from a multitude of viruses in a biological sample.

A twenty first embodiment includes any of the first through the twentieth embodiments wherein the experimental protocol for isolating the virus can involve sequencing one or more virus species of interest without the use of primers by directly sequencing the RNA species in a biological sample without any amplification step.

A twenty second embodiment includes any of the first through the twentieth embodiments wherein the experimental protocol for characterizing the virus involves sequencing one or more virus species of interest and the sequencing step includes an amplification step.

A twenty third embodiment includes any of the first through the twenty second embodiments where sequence data required for comparative purposes is saved locally, or remotely.

In a twenty fourth embodiment includes any of the first through the twenty third embodiments wherein sequence data for up-oad is stored locally before it is uploaded or uploaded directly to a remote data base.

### DESCRIPTION

Point-of-care diagnostic systems includes devices that are physically located at the site where patients are tested and sometimes treated to provide quick results and highly effective treatment. Point-of-care devices have the potential to reduce health care costs by providing rapid feedback on disease states and information and help in diagnosing patient disorders and/or infections while the patient is present with potentially immediate referral and/or treatment. Unlike gold standard laboratory-based testing for disorders and/or infections, point-of-care devices enable diagnosis close to the patient while maintaining high sensitivity and accuracy aiding efficient and effective early treatment of the disorder and/or infection.

The global spread of COVID-19 galvanized the need to develop tests and treatments for SARS-CoV-2. The sheer number of infected individuals and this virus' ability to evolve has also made in imperative that its variants can be identified, tracked, and treated. Tests for this virus include both SARS-CoV-2 protein tests (PTs) and nucleic acid tests (NTs). The current gold standards for COVID-19 diagnostic kits are based on PCR technologies due to their exceptional reliability compared to other techniques.

Rapid Antigen Detection (RAD) PTs are common point-of-care tests that return results in minutes compared to the hours required for PCR. However, RAD PTs suffer from considerably lower sensitivity and specificity than PCR methods Antibody PTs can reveal if an individual was infected months ago, something PCR tests cannot do, but can return false negative results if the individual was infected very recently

Next Generation Sequencing NTs were the first to identify SARS-CoV-2 and can identify new strains but are less scalable and cost-effective than PCR RT-LAMP isothermal amplification protocols require less time, materials and expertise than PCR; however, primer design is more complex than for PCR and PCR sensitivity is slightly higher [26-28]. Another NT approach uses CRISPR to detect amplicons generated by isothermal amplification; this combined technique offers similar results to PCR kits but is limited by reagent availability [29-30].

The utility of PCR test kits is corroborated by their use throughout the US. According to the Centers for Disease Control and Prevention (CDC), 180 million PCR tests have been performed in the US]. These tests come from a pool of 183 PCR test kits granted emergency use authorizations from the US Food and Drug Administration]. Given the importance of PCR diagnostic kits to the US COVID-19 testing infrastructure, a number of organizations and databases offer resources to guide PCR primer design.

Informed primer design is indispensable to successful PCR tests. The CDC made its list of real time PCR primers public in January 2020 and the World Health Organization (WHO) similarly published primer pairs with multiple SARS-CoV-2 gene targets. A number of online databases also provide reliable primers for SARS-CoV-2. The Arctic database holds an updated pool of SARS-CoV-2 primers and also features primer tiling across the entire viral genome [35]. Another database, MRPrimerV, also features primer sets for a range of viruses including SARS-CoV-2 [36]. The ViPR database also supports a primer design tool that uses the Primer 3 algorithm to generate coronavirus-specific pairs [37].

Although the resources above provide useful information, each could be further improved. For instance, the CDC/WHO primer pool is relatively small with less than 100 pairs. The Arctic database Artic contains a higher number of primers; however, it is not indicated whether these primers are specific only to SARS-CoV-2. The MRPrimerV database offers primers for SARS-CoV-2 and several other viral species [36]. Finally, the ViPR database offers a tool for PCR primer design but is not a dedicated primer database. It is expected that the breath, accuracy, and accessibility of such data bases will improve with time, accordingly, various embodiments of the invention will be able to use so improved data bases,

Typically, the most sensitive assays require the support of a technologically sophisticated and capital-intensive healthcare infrastructure. Under current methods, patient samples taken at the point-of-care must be transported to a laboratory that maintains the equipment and personnel required to perform the actual test. Low resource settings simply do not have access to such facilities, which precludes these areas from having access to the most sensitive diagnostics. Some of the inventive methods disclosed herein include the use of devices and/or systems that offer on-site analysis and allow for use of highly sensitive diagnostics in settings where the healthcare infrastructure is less developed and/or where the high number of infections make it difficult to process high numbers of samples.

One effective means of identifying the virus includes the extraction of viral RNA from sample obtained from patients and the storage of sample material. Individually or in tandem these steps may be coupled with the whole genome sequencing (WGS) of viral pathogens. While PCR-based detection methods focus on small amplicons, viral WGS applications require RNA of high quality and integrity for adequate sequence coverage and depth. Efficient and reproducible RNA extraction is an important factor in the detection and sequencing of pathogenic viruses in a clinical laboratory setting. Automated extraction platforms are routinely used to improve extraction efficiency and to ensure consistent results in diagnostic laboratories. There have been many studies evaluating the performance of various automated and manual extraction platforms, and the choice of extraction platform has been shown to have a major impact on the reliability of results for diagnostics. Based on findings of all these studies and current method of WGS to study metagenomics, platforms using EZ1 Advanced XL (Qiagen) or similar approaches appear to perform better. EZ1 is fully automated system to isolate DNA or RNA from various bio samples. It can handle 14 samples at a time, saving time and risk of exposure to infectious samples. EZ1 can generate samples of better quality and yield. Such samples after a series of library preparation steps could be sequenced using sequencing platforms available from Illumina, Pacific Biosciences and Oxford Nanopore Technologies. Unlike the traditional sanger sequencing methods which generate Short Read Sequencing (SRS) data, recently developed Long Read Sequencing (LRS) approaches from all of the new generation platforms are synthesis independent and can generate cDNA sequencing or direct RNA sequencing reads at single molecule resolution. Hence, it is an advantage over current short read sequencing (SRS) methods to employ these next generation sequencing methods for studying ensemble of viral genomes i.e., viromes in a clinical sample. To sequence RNA using SRS methods, RNA should be fragmented and converted to cDNA before sequencing. Short fragments are used to generate whole genome sequences using computational tools knowns as assemblers. This method is limited by two major concerns, A) errors introduced by reverse transcriptase enzyme (rt) while converting RNA into cDNA molecules and B) quality of resulting assembled genomes as they cannot differentiate between reads of repetitive regions and homopolymer sequences. In contrast, LRS methods can be synthesis independent and can generate reads of any length, making it possible to sequence entire genome in one read or a smaller set of reads, which can then be used to not only assemble the genome but to also study the presence and evolution of strains occurring in a clinical sample. Combining a correct isolation method with a WGS approach and developing a state of the art computer software specifically tailored for detecting the presence of the viruses, can reduce sequencing time and data analysis time which is important for enabling rapid detection of viral agents from clinical samples. The inventive methods provide a real time scalable end to end sequencing to data analysis platform integrated with visualizations for detection, diagnosis, estimation, surveillance of the viral burden and its evolution, from clinical isolates of body fluids such as nasopharyngeal, saliva and oral swabs.

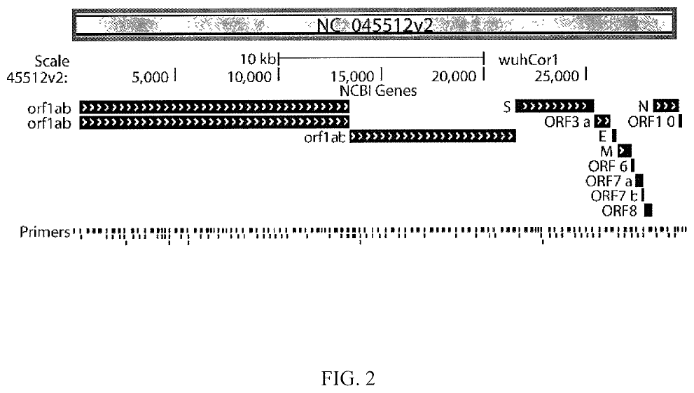

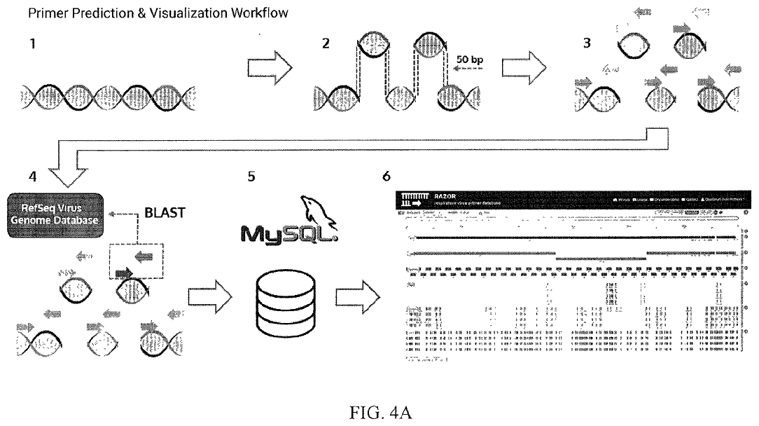

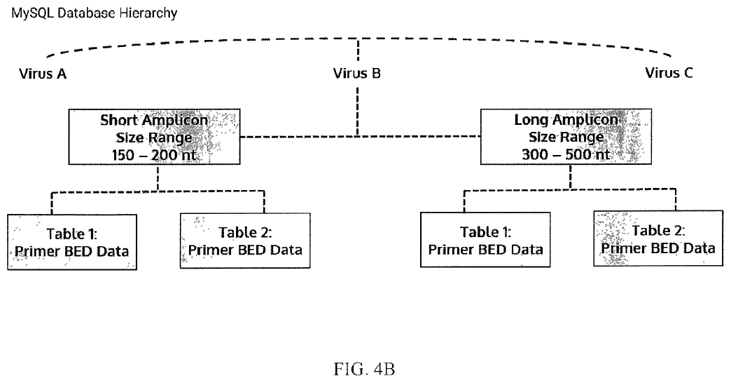

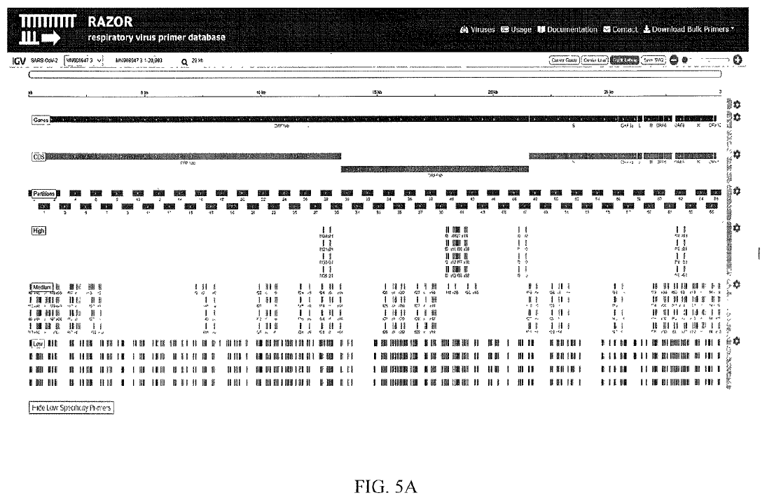

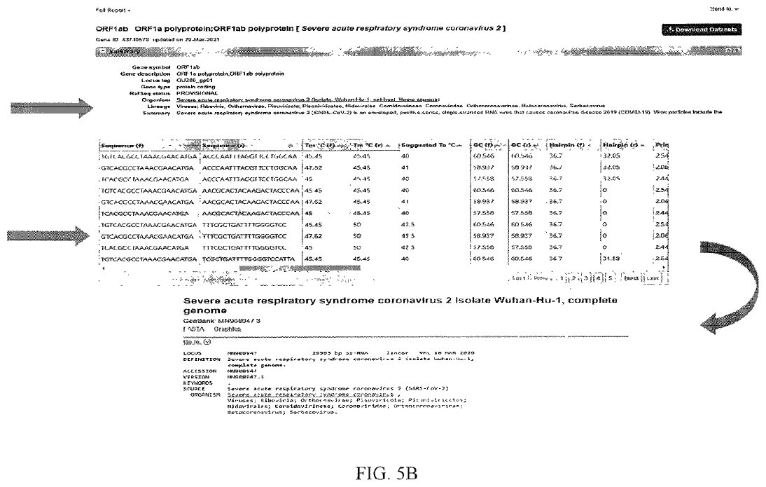

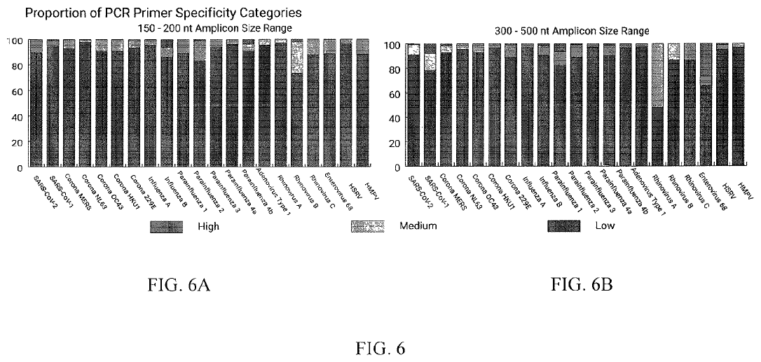

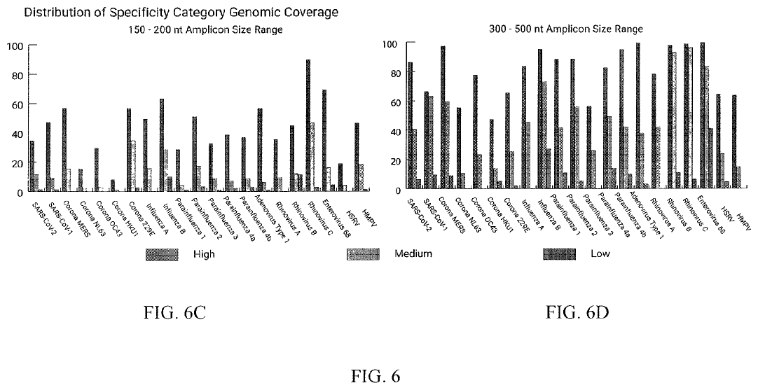

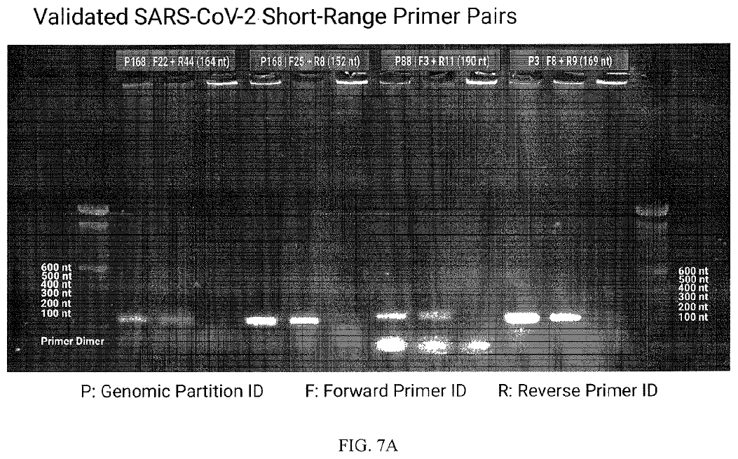

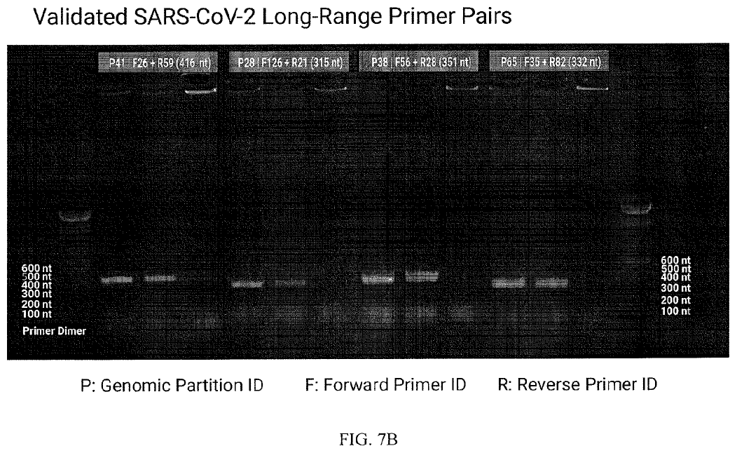

For detection and quantitative estimation of viral genome including SARS-CoV-2 in infected host cells, the inventive method includes efficient, novel and high-throughput RNA isolation steps combined with a long read sequencing method such as those resulting from sequencers of Oxford Nanopore Technologies, Pacific Biosciences as well as short read sequencers from Illumina to develop an automated computational software for real time monitoring, data analysis, visualization and live reporting at individual steps. A fully automated and robust platform for the diagnosis of viral infection in multiple samples and their abundance in real time is implemented after viral RNA isolation from human body fluids. Some embodiments of the invention streamline the end-to-end library preparation steps of 96 nasopharyngeal or saliva samples using viral RNA and or DNA isolated via available viral RNA or DNA extraction kits, to generate barcoded long read sequencing data by employing massive high throughput robotic technology (such as Hamilton Company-NGS workstation). Briefly, in some embodiments the specimens collected from naso/oropharyngeal swab or other body fluids will be contained in viral transport medium. The viral RNA will be isolated from the swab/fluids using Zymo Research Quick DNA-RNA Viral Kit. A panel of primers specific for a wide range of respiratory viruses including SARS-CoV2, providing genomic coverage at different levels of specificity based on their extent of conservation across viral genomes (illustrated in FIGS. 2, 4, 5 and 6) is developed as part of this application. Primer panels from such inhouse database (https://sysbio.informatics.iupui.edu/primer_project/razor/) or an different data base which includes the same, or similar information provide the ability to detect either a single viral genome in a sample or a combination of viruses when a customized primer panel is selected for the group of viruses. Such panels can facilitate the batch amplification of viral fragments either in size range of 150-200 nt amplicons (short amplicons) or 300-500 nt (long amplicons) (FIGS. 4 and 6) resulting in sequencing of RNA/DNA fragments in each specimen and can be customized to detect the presence or absence of a multitude of viruses (in combination depending on the specific clinical need) for at least 96 samples in a single sequencing run on a benchtop sequencer from Oxford Nanopore Technologies (ONT).

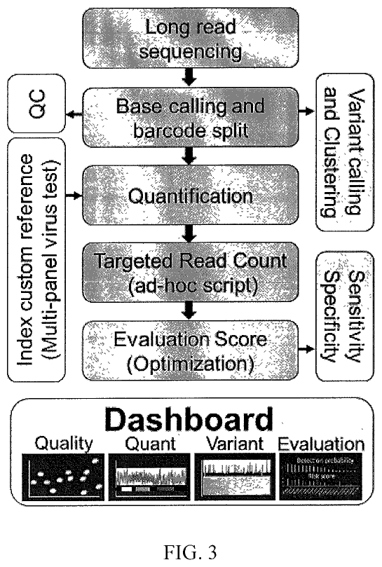

In some embodiments target specific primers can be barcoded with a PCR Barcoding Expansion 1-96 kits (EXP-PBC096) (or subsequent versions of such kits) from ONT that enable the multiplexing of RNA/DNA samples for batch amplification. RNA samples, amplified with the barcoded primers in multiplex-PCR platform, will be pooled as per the manufacturer's instruction. These pooled barcoded amplicons will be loaded in MinION Mk 1B or Mk1C or similar long read sequencing platforms. Such sequencing protocol with barcoding for viral enriched samples from human specimens can be replaced by other LRS sequencing methods available from Pacific Biosciences and Illumina to increase the scale of the number of samples that can be screened using long read sequencing. It is also important to note that in the above pipeline, primer based amplification step can be completely removed since the samples are enriched for the presence of viral titers via kits such as Zymo Quick DNA-RNA Viral kit or Qiagen's QIAamp Viral RNA Mini Kit, to either perform direct cDNA sequencing of the amplicons or employ direct RNA-sequencing method when the sequencing platform such as ONT enables it. The result of these proposed steps enables screening of ≥96 samples at the same time via a single amplicon sequencing experiment using targeted amplification of either specific groups of viral genomes of interest (defined as viral panel based on the primers used) or denovo sequencing of the complete virome present in a human sample without the need for primer based amplification. Proposed pipeline of steps enable sequencing of the viral genomes present in a sample in high-throughput mode and hence the resulting data provides information on their abundance, mutations, evolution and origin of strains being identified, which is not possible with current rt-PCR or other diagnostic tests that are commonly employed. More importantly, proposed methods are massively scalable for a large number of samples and can result in real time monitoring Point of Care (PoC) viral diagnostic tests, if employed with benchtop/handheld sequencers such as MinION Mk 1B or Mk1C or smidgION from ONT. Embodiments of the invention employ the resulting long read sequencing data for developing a series of visualization tools by integrating both publicly available open access software and in house developed tools as described below, to generate a diagnostic platform of viral presence, abundance estimation, mutations, serotyping and evolutionary analysis as a one stop software for viral diagnostics and surveillance from sequencing data. In particular, for long read benchtop sequencers embodiments of the invention include monitoring and visualization tools for each step during the sequencing in real time by using the data resulting from long read sequencing. Also, the abundance of each viral fragment amplified with the barcoded primers will be monitored in real time as the data is being generated to present a dashboard with the presence, abundance of the viral titers and accompanying statistics (FIG. 3). For sequencing platforms that do not provide data in real time, such integrated software platform will naturally provide dashboards of the final resulting datasets on any computer with windows, macs and linux. Platform dependencies will be handled by providing pre-packaged containers such as docker for easy portability across systems.

Optionally, the inventive computational pipeline may include a dashboard on top of ONT sequencers (which are connected to a computing module with an operating system) to monitor each step including the in-built base calling, customized to perform base calling and barcode splitting in real time as well as to stop the sequencer if needed. Since barcodes correspond to different human specimens, data will be employed to show the presence/abundance, variants, closest strains, phylogenetic relationships with other viruses that are already available from the NCBI reference viral genome database. The dashboard will also provide a real time mean read quality, abundance, length distribution and variation across samples. A schematic workflow of proposed computation pipeline and corresponding visualization tools is shown in FIG. 3. Briefly, nanopore based sequencing of the amplified viral RNA fragments will be base called in real time along with enabled barcode split mode, using base calling algorithms available on board the machine or from the sequencing manufacturer and monitored for live read quality and length distribution. High quality and barcode deconvoluted cDNA/RNA sequencing reads will be processed for variant calling in real time using NanoVar. Further, a rapid alignment or alignment free mapping tool will be employed to estimate the abundance of each region. For instance—Sailfish will be employed to estimate the abundance of post-processed amplicons against the targeted viral genome/s (i.e. after indexing the reference fasta sequence of the genomes) and the read counts will be extracted using ad-hoc scripts to provide the end user with a dashboard display/plots showing for each sample coverage of the reads along the viral genome/genomes in the viral panel, estimated abundance score, mutations as well as confidence score for associating the sample with a specific set of viral strains present in the sample. Normalized read coverage will be computed for each viral gene across all the samples and provided as a visualization on the dashboard. A comprehensive monitoring of the normalized coverage for all the viral genes illustrated on the dashboard will be evaluated in real time to provide an estimated virus specific detection score and its pathogenicity score based on prior annotations of the virulence levels in public databases. Additionally, the dashboard will enable the profiling of mutational landscape of virus strain and its origin around the world by comparing with the open source viral strain databases. The dashboard will also provide metrics such as confidence level with which each sample is annotated for the presence of a virus along with a comprehensive summary of the virus detection probability and risk score for all the patient samples sequenced. All of these steps will be achieved in real time for all the samples being processed as the sequencer is generating the data for benchtop real time sequencers. For sequencers which do not provide this option of real time monitoring and processing of the data, the software can be deployed for post-processing and analysis to provide the results to the user by providing the data resulting from the sequencer with barcoding information. This pipeline and integrated toolkit will enable the rapid diagnosis of viral RNA/DNA at scale, along with the real-time detection of specific strains prevalent in a geographical site and allow comparison with other strains around the world that are sequenced so far, helping iterative improvements in surveillance as the database of viral genomes increases and facilitate vaccine design efforts for novel and emerging viruses.

Embodiments of the present invention provide a step by step framework for an automated library preparation protocol for facilitating pooled multi-sample cDNA and RNA long read sequencing of viral enriched RNA/DNA samples from human body fluids. Such a multi-step protocol will enable high-throughput screening of ≥96 nasal/oral/saliva swab/fluid samples combined with multiplexing-PCR, long read sequencing and developing an automated pipeline embedded with a dashboard for rapid diagnosis, analytics and monitoring of virus pathogenicity and surveillance in real time across human specimens on benchtop sequencers. The software toolkit/framework can also be used as a standalone suite of tools and will work on any long-read sequencing datasets emerging from viral isolations from clinical samples of the body fluids to facilitate viral load, genome analysis, evolution and origin. Some of the advantages of some embodiments of the present invention, individually or in various combinations, include but are not limited to the:

1. Ability to develop a custom panel of broad range primers that enables the detection and targeted DNA/RNA fragment amplification in size ranges 150-200 nt, 300-500 nt or ≥400 nt for a wide range of viruses of clinical interest to facilitate design and targeted sequencing of specific viral panels. The inventive method has been applied to SARS-CoV2 targeted sequencing in clinical samples of nasopharyngeal and oropharyngeal swab specimens to demonstrate the success of the proposed viral panel for accurate detection of the viral presence.

2. Ability to develop two variants of pooled and barcoded long read sequencing protocols for viral enriched samples from body fluids namely A) primer independent amplification free cDNA sequencing protocol and B) reliable PCR-free approach using direct RNA sequencing protocol, accompanied by automated and integrative long read data analysis pipelines for detection of viruses, with real time mapping and visualization software where the sequencers permit real time data analysis.

3. Ability to deploy these experimental protocols and computational frameworks on any of the Oxford Nanopore Technology based sequencers to facilitate real-time long read sequencing and the resulting data interpretation, for clinical viral diagnostics from body fluids.

4. Ability to deploy the proposed computational pipelines, artificial intelligence algorithms, and mapping and visualization display software with the above described functions (FIG. 3), to summarize the results in real time using the long-read sequencing datasets for viral enriched samples. These tools can either be applied to those resulting from benchtop sequencers or for post-processing on other sequencing systems, to rapidly annotate the presence and abundance of viral strains (SARS-CoV2 virus is shown as an example in this application) for detailed understanding of the prevalence of various viral species present in a clinical sample along with probabilistic scores for their enrichment and risk scores for pathogenicity.

5. Ability to detect the genotypes/serotypes of viral species present in a clinical sample and to be able to de novo detect new strains/species of viral genomes significantly emerging in a population from clinical sequencing to enable surveillance, national database collection and vaccine development efforts.

6. Ability to quantify the level of infection for each viral specie in a clinical sample based on the resulting sequencing data in addition to a simple positive and negative test outcome, enabling the simultaneous diagnostics of multiple viral species in a sample. Thus, providing a summary report to an end-user to enable real-time decisions on the level and impact of infection in a patient sample right in the clinic or field where the instrument is deployed.

7. Ability to identify new viruses or variants of known virus by real time comparisons of viral nucleic acid sequences identified in a sample recovered from patients with sequences stored in internal or shared databases comprised of previously identified sequences.

8. Ability to quickly share nucleic acid sequence information on new viruses or variants identified in the given region.

### EXPERIMENTAL

### Experiment 1

We developed a respiratory viral primer database ‘RAZOR’ and used it to provide high-quality PCR primers for 21 human respiratory viruses, including SARS-CoV-2. This database was used to predicted primers corresponding to two amplicon size ranges (150-200 nt & 300-500 nt) which can be applied to either real-time or traditional PCR protocols. The primer pairs are binned into at most three distinct specificity categories (High, Medium, & Low) depending on the number of virus genomes targeted. Results are shown in an event driven IGV interface with several options for querying, filtering and downloading data. RAZOR also supports community-driven collaboration where experimentalists can submit validations of predicted primers for all users to view.

### Materials & Methods

### Data Sources:

Viral Genomes Reference genomes for 21 human respiratory viruses were downloaded in FASTA and GenBank format from the NCBI Nucleotide database. In the case of viruses with segmented genomes (Influenza A & B), each segment was treated as a distinct Nucleotide entry with segment-specific sequence files. The list of the respiratory viruses and corresponding NCBI accession identifiers is provided in Table 1 along with an estimate of the total number of primers developed for each viral genome.

TABLE 1
Table summarizing the various respiratory viral genomes
included in this embodiment, NCBI accession numbers of
their genomic sequences, genome size and the total number
of designed primers satisfying the criteria described.

| | | | |
|---|---|---|---|
| MN908947.3 | HCoV SARS-CoV-2 | 29903 | 202873 |
| AY714217.1 | HCoV SARS-CoV-1 | 29727 | 197688 |
| NC_038294.1 | HCoV MERS | 30111 | 237348 |
| NC_005831.2 | HCoV NL63 | 27553 | 29030 |
| NC_006213.1 | HCoV OC43 | 30741 | 46434 |
| NC_006577.2 | HCoV HKU1 | 29926 | 38820 |
| NC_002645.1 | HCoV 229E | 27317 | 41662 |
| AC_000017.1 | Adenovirus Type 1 | 36001 | 230334 |
| NC_039199.1 | Human Metapneumovirus (HMPV) | 13350 | 46130 |
| NC_001803.1 | Human Respiratory Syncytial Virus | 15191 | 23220 |
| | (HSRV) | | |
| NC_038308.1 | Human Enterovirus Type 68 (HEV) | 7367 | 83890 |
| NC_038311.1 | Rhinovirus A | 7137 | 38674 |
| NC_038312.1 | Rhinovirus B | 7208 | 91140 |
| NC_038878.1 | Rhinovirus C | 6944 | 116112 |
| NC_003461.1 | Parainfluenza 1 | 15600 | 60218 |
| NC_003443.1 | Parainfluenza 2 | 15646 | 95496 |
| NC_001796.2 | Parainfluenza 3 | 15462 | 50966 |
| NC_021928.1 | Parainfluenza 4a | 17052 | 72676 |
| MN306032.1 | Parainfluenza 4b | 17384 | 64034 |
| NC_007366.1- | Influenza A | 13627 | 156076 |
| NC_007373.1 | | | |
| NC_002204.1- | Influenza B | 14452 | 177488 |
| NC_002211.1 | | | |

BLAST Database The most recent release of the NCBI Ref Seq viral genomes database was downloaded from the NCBI FTP server (https://ftp.ncbi.nlm.nih.gov/refseq/release/viral). The “makeblastdb” command was used to generate a local BLAST database from the downloaded file.

### Computational Resources:

Indiana University Carbonate Carbonate is an Indiana University large-memory computer cluster of 80 compute nodes. Each general-purpose node is a Lenovo NeXtScale nx360 m5 server equipped with two Intel Xenon E5-2680 v3 12-core CPUs, four 480-GB SSDs and 256 GB of RAM [38]. Carbonate is designed for intensive tasks (high memory overhead) and was utilized to generate and filter volumes of primer predictions.

IUPUI Lab Servers RAZOR uses two lab-owned servers. Each server contains 64 8-core AMD Opteron 6276 CPUs. One hosts the database webpages and the other hosts a symbolically linked MySQL database that holds the primer records.

### Construction of PCR Primers:

Primers in RAZOR were constructed with a custom Python 3 (3.8.6) pipeline scaled to the Indiana University Carbonate cluster. The pipeline was comprised of a series of modules/steps: genome partitioning, primer prediction, primer specificity analysis, primer pair assembly, pair filtering, and result storage. FIG. 4(A) provides an overview of the prediction pipeline.

Genome Partitioning All downloaded viral genome FASTA sequences were split at regular intervals to create a series of overlapping, n-length partitions. The genomic partitions defined a search space for amplicons bounded by the partition genomic coordinates. Partition length was decided by amplicon size range (n=200 nt for short range, n=500 nt for long range) and each partition overlapped its neighbor(s) by 50 nt.

Primer Predictions A local distribution of Primer3 [39] was used to generate amplicons and primers for each partition of a viral genome. Changes to the default Primer3 parameters are listed: PRIMER_PRODUCT_SIZE_RANGE was set at 150-200 for short-range partitions and 300-500 for long-range, SEQUENCE_TEMPLATE was set as partition sequences, and both PRIMER_PICK_LEFT_PRIMER and PRIMER_PICK_RIGHT_PRIMER (option to generate forward and reverse primers) and PRIMER_MAX_NS_ACCEPTED (maximum number of unknown bases in a primer sequence) were set to 1. After each Primer3 run, primer IDs, sequences, melting temperatures (Tₘ), GC content (% GC) and Primer3 quality scores were appended to a shared .tsv file.

Primer Specificity Analysis A local distribution of BLAST 2.3.0 [40] was used to compare each primer from the previous step's .tsv file to a RefSeq viral genome database (9277 genomes total). Primers with 1 BLAST hit were placed in a Low Conservation group, primers with 2-5 blast hits in a Medium Conservation group, and primers with 6-10 hits in a High Conservation group. Following the sorting, the shared primer .tsv file is partitioned into three separate .tsv files which were populated with primers appropriate to each group.

Primer Pair Assembly For all new .tsv files, genomic coordinates were scanned to find pairs of forward and reverse primers for genomic partitions. Actual amplicon product size was computed as the distance between the start of the forward primer and end of the reverse. Annealing temperature Tₐ was computed as 5° C. less than the average of the primer pair's Tₘ values. Both primers in a pair generally belonged to the same conservation category; however, Low Conservation primers sometimes lacked a partner (which was assigned instead to the Medium or High conservation categories). For such cases, appropriate partners from the Medium and High categories were copied over to complete the pair.

Pair Filtering A final filtering step was implemented to ensure that all primer pairs will be useful in PCR experiments. Primer pairs with a difference in melting temperature greater than 5° C. were discarded as well as pairs where the highest Tₘ value was at least 10° C. higher than the lowest primer stable hairpin melting temperature.

Result Storage: RAZOR primer data was stored in a MySQL 5.1.73 database through the pymysql connector. All viral genomes contained data for short and long amplicon size ranges. Two tables were created for each size range: one containing individual primer BED information and other containing primer pair sequences and metadata. The general database hierarchy is represented in FIG. 4(B).

Primer Analyses: Two analyses were performed on the predicted primers: (i) primer specificity category distribution & (ii) genomic coverage calculation. The conservation category distribution analysis was performed by calculating the percentage of each category for each genome at a certain amplicon size range (i.e. SARS-CoV-2 category distribution for long size range: 86.20% High, 40.70% Medium, 6.32% Low). The genomic coverage analysis was performed for each genome at each amplicon size range by finding all genome partitions with primer data present, obtaining the largest amplicon size for each of these partitions, and then dividing the sum by the length of genome. This calculation is represented by the formula below, where N denotes the number of genome partitions with primer data, n denotes a single partition and L denotes length in nt:

$$Genomic Coverage = \frac{{\sum }_{0}^{N} ( max ( L amplicon partition ∈ n ) )}{L genome} \times 100 \%$$

Following the two analyses, an extra table was added to the database. Results for the distribution and genomic coverage analyses were inserted; each row represented a distinct viral genome with both Short and Long amplicon size range data.

Downloadable Files: A Python 3 script was used to extract primer information for all of each viral genome's partitions, convert BLAST hit data into a more human-readable format, and save the information to compressed comma-separated values (csv.gz) and Excel spreadsheet (xlsx.gz) files.

### Experimental Validation: Selected SARS-CoV-2 Primer Pairs

To confirm the utility of predicted PCR primer, eight primer pairs for SARS-CoV-2 were selected for experimental validation. Four pairs correspond to the short amplicon range and the others correspond to the long range. The primer IDs are shown in Table 2 below.

TABLE 2

| | | | For- | | | Re- | |
|---|---|---|---|---|---|---|---|
| | | For- | ward | | Re- | verse | |
| | Ampli- | ward | Pri- | | verse | Pri- | |
| Gene | con | Pri- | mer | SE | Pri- | mer | SE |
| Tar- | Size | mer | se- | ID | mer | se- | ID |
| geted | Range | ID | quence | NO: | ID | quence | NO: |
| ORF1ab | 150-200 | f8 | GAGCT | 1 | r9 | GATCG | 9 |
| | | | GGTAG | | | GCGCC | |
| | | | CAGAA | | | GTAAC | |
| | | | CTCG | | | TATG | |
| ORF1ab | 150-200 | f3 | GTAGC | 2 | r11 | TTGGC | 10 |
| | | | TTGTC | | | CGTGA | |
| | | | ACACC | | | CAGCT | |
| | | | GTTTC | | | TGACA | |
| N | 150-200 | f22 | AACTC | 3 | r8 | TCTGC | 11 |
| | | | AAGCC | | | ATGAG | |
| | | | TTACC | | | TTTAG | |
| | | | GCAGA | | | GCCTG | |
| | | | | | | A | |
| N | 150-200 | f25 | ACTCA | 4 | r44 | CTGCA | 12 |
| | | | AGCCT | | | TGAGT | |
| | | | TACCG | | | TTAGG | |
| | | | CAGA | | | CCTGA | |
| ORF1ab | 300-500 | f126 | CTTGT | 5 | r21 | GTAGA | 13 |
| | | | GCTGC | | | CGTAC | |
| | | | CGGTA | | | TGTGG | |
| | | | CTAC | | | CAGC | |
| ORF1ab | 300-500 | f56 | TGCTA | 6 | r28 | CTAGT | 14 |
| | | | TTGGC | | | GTGCC | |
| | | | CTAGC | | | CTTAG | |
| | | | TCTCT | | | TTAGC | |
| | | | ACT | | | A | |
| ORF1ab | 300-500 | f26 | ACTTC | 7 | r59 | TGGAC | 15 |
| | | | CTTGG | | | AGCTA | |
| | | | AATGT | | | GACAC | |
| | | | AGTGC | | | CTAGT | |
| | | | GT | | | | |
| N, | 300-500 | f35 | ACGTG | 8 | r82 | CTGCA | 16 |
| ORF10 | | | GTTGA | | | TGAGT | |
| | | | CCTAC | | | TTAGG | |
| | | | ACAG | | | CCTGA | |

In order to check primer quality and off target amplifications, we used three different COVID samples to run PCR and gel electrophoresis to check amplification bands. Remnant nasopharyngeal and oropharyngeal swab specimens collected from COVID-19 patients were collected in viral transport media. RNA was isolated using Zymo Research Quick-DNA/RNA Viral Kit (D7021) as per manufacturer instructions. RNA was reverse transcribed into cDNA using SuperScript™ IV Reverse Transcriptase (18090010). A 20-μl reaction was set up containing 2 μl of RNA, 10 μl of SapphireAmp® Fast PCR Master Mix, 1 ul of Forward primer (10 uM), 1 ul of Reverse primer (10 uM) and 6 ul water. Thermal cycling was performed by 95° C. for 3 min and then 30 cycles of 95° C. for 15 s, 55° C. for 30 s, 65° C. for 1 minute and termination at 65° C. for 2 minutes. Samples were run on a 1% agarose gel and amplicons were captured.

### Experiment 2

We determined the prevalence of specific strains of SARS-CoV-2 and mapped their spread through the population of the State of Indiana in the early stages of the COV-19 pandemic. Experimental protocols, computational pipelines and corresponding inferences are summarized below. This body of work is accomplished using benchtop real time sequencing of COVID positive samples.

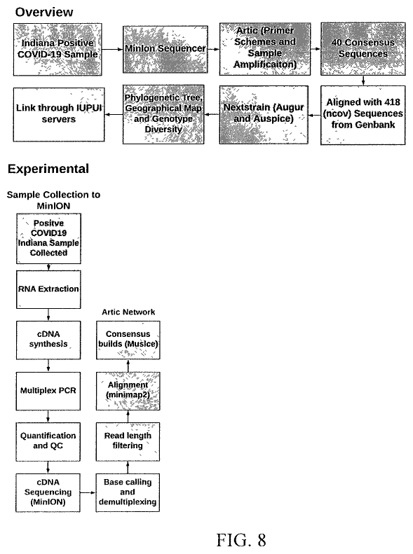

Experimental and computational framework for genotyping SARS-CoV-2 in Indiana samples. RNA was extracted from each of the Indiana samples as described in methods and then conducted cDNA synthesis, multiplex PCR, quantification and quality control steps. cDNA sequencing was operated by the MinIon Oxford Nanopore sequencer. Base calling and demultiplexing were used to achieve enough sequencing depth per sample, for the analysis. Read length filtering was executed to ensure only quality read lengths were included. Since long read sequencers such as MinION often produce longer chimeric reads along with the actual reads with expected length, we included the reads of length ranging from 300 to 700 bp in our analysis. The Minimap2 program was used for the alignment process in mapping the (Li, 2018). Since each sample went through the process multiple times, Muscle was used to build consensus sequences for each the time the positive sample went through the (Edgar, 2004). The Artic Network was used to create consensus sequences for 40 positive COVID-19 samples (FIG. 8). The Phylogenetic tree was grouped by genomic diversity and geographical location. Genomic diversity was used to infer mutation sites among the samples. Geographical location was used to determine which countries had the most similar sequences to the samples from Indiana. Finally, the sequences were phylogenetically analyzed through the Nexstrain system (FIG. 8). This pipeline has been set up for data collection so that our lab can collect, sequence, and display sequences on a web browser.

The phylogenetic analysis shows that 39 of the Indiana samples are in the G-type, while 1 Indiana sample is located in the D-type). Based on a Fisher's exact test on samples with Glycine or Aspartic acid and from Indiana or not from Indiana. Our result shows a significant enrichment of Indiana samples for G-type (p-value: 1.63e-06 and the odds ratio: 21.73).

A Majority 65% of the Indiana Samples had a branch confidence percentage of 100% Indiana. Inside the United States, the SARS-CoV-2 sequences were most similar to sequences from Virginia, Michigan, and other strains from Indiana. Outside the United States, the SARS-CoV-2 sequences are most similar to sequences from Victoria Table 3

TABLE 3

| | | | | | | Branch leading |
|---|---|---|---|---|---|---|
| | | | | | | to Samples |
| Sample | Sample | CT | | Gen- | Signs & | Division |
| Number | ID | Value | Age | der | Symptoms | (confidence) |
| 1 | Pos- | 30.7 | 70 | f | cough, | Indiana (100%) |
| | 91 | | | | dehydration | |
| 2 | Pos- | 37.09 | 63 | m | fever, | Indiana (100%) |
| | 92 | | | | cough | |
| 3 | Pos- | 29.34 | 56 | m | fever, | Indiana (99%) |
| | 95 | | | | cough, | Victoria (1%) |
| | | | | | shortness | |
| | | | | | of breath, | |
| | | | | | fatigue | |
| 4 | Pos- | 24.62 | 37 | m | fever, chills, | Indiana (100%) |
| | 107 | | | | aches, | |
| | | | | | headache, | |
| | | | | | congestion | |
| 5 | Pos- | 22.14 | 36 | f | cough, | USA (35%), |
| | 108 | | | | headache, | New York (32%), |
| | | | | | chest | Indiana (21%), |
| | | | | | discomfort, | Victoria (9%) |
| | | | | | shortness | |
| | | | | | of breath, | |
| | | | | | sore throat | |
| 6 | Pos- | 23.03 | 13 | m | fever, | Indiana (100%) |
| | 163 | | | | headache | |
| 7 | Pos- | 19.07 | 50 | f | fever, | Michigan (98%), |
| | 164 | | | | aches, | Victoria (1%), |
| | | | | | chills, | Indiana (1%) |
| | | | | | nausea, | |
| | | | | | emesis | |
| 8 | Pos- | 27.25 | 48 | m | shortness | Indiana (100%) |
| | 165 | | | | of breath, | |
| | | | | | fever, | |
| | | | | | cough, | |
| | | | | | aches, chills | |
| 9 | Pos- | 28.49 | 44 | f | headache | Indiana (100%) |
| | 166 | | | | | |
| 10 | Pos- | 16.58 | 30 | f | cough, | Indiana (100%) |
| | 167 | | | | fever, chills, | |
| | | | | | congestion, | |
| | | | | | sore thorat, | |
| | | | | | headache | |
| 11 | Pos- | 22.78 | 53 | m | fever, | Indiana (100%) |
| | 120 | | | | diarrhea, | |
| | | | | | cough, | |
| | | | | | shortness | |
| | | | | | of breath, | |
| | | | | | chest | |
| | | | | | discomfort | |
| 12 | Pos- | 24.1 | 27 | f | fever, | Victoria (100%) |
| | 121 | | | | cough, | |
| | | | | | shortness | |
| | | | | | of breath, | |
| | | | | | fatigue, | |
| | | | | | aches, | |
| | | | | | diarrhea | |
| 13 | Pos- | 18.22 | 83 | f | fall, altered | Indiana (100%) |
| | 170 | | | | mental | |
| | | | | | status, | |
| | | | | | cough | |
| 14 | Pos- | 20.39 | 39 | f | cough, sore | Victoria (96%), |
| | 125 | | | | throat, | Indiana (3%) |
| | | | | | aches | |
| 15 | Pos- | 17.58 | 43 | f | unknown | Victoria (96%), |
| | 44 | | | | | Indiana (3%), |
| | | | | | | Michigan (1%) |
| 16 | Pos- | 41.64 | 67 | f | fever, | Indiana (98%), |
| | 130 | | | | cough | Victoria (2%) |
| 17 | Pos- | 19.67 | 66 | f | shortness | Indiana (100%) |
| | 131 | | | | of breath, | |
| | | | | | cough, | |
| | | | | | syncope, | |
| | | | | | rhinorrhea | |
| 18 | Pos- | 19.26 | 56 | f | aches, | Indiana (100%) |
| | 54 | | | | headache, | |
| | | | | | fatigue | |
| 19 | Pos- | 25 | 96 | f | cough | Indiana (100%) |
| | 56 | | | | | |
| 20 | Pos- | 34.28 | 61 | m | unknown | Indiana (99%), |
| | 58 | | | | | Ahemedabad |
| | | | | | | (0%) |
| 21 | Pos- | 38.12 | 50 | m | shortness | Indiana (100%) |
| | 59 | | | | of breath, | |
| | | | | | cough, | |
| | | | | | fever | |
| 22 | Pos- | 34.32 | 45 | f | shortness | Indiana (100%) |
| | 60 | | | | of breath, | |
| | | | | | aches, | |
| | | | | | nausea, | |
| | | | | | emesis | |
| 23 | Pos- | 27.83 | 55 | f | cough, | Indiana (100%) |
| | 61 | | | | shortness | |
| | | | | | of breath, | |
| | | | | | fever, | |
| | | | | | emesis, | |
| | | | | | aches, | |
| | | | | | headache, | |
| | | | | | congestion | |
| 24 | Pos- | 24.34 | 79 | f | cough, | Indiana (99%) |
| | 187 | | | | weakness, | Victoria (1%) |
| | | | | | fever, | |
| | | | | | fatigue, | |
| | | | | | sore throat | |
| 25 | Pos- | 24.99 | 53 | f | aches | Indiana (100%) |
| | 188 | | | | | |
| 26 | Pos- | 18.96 | 47 | m | fever, | Indiana (100%) |
| | 189 | | | | diarrhea, | |
| | | | | | abdominal | |
| | | | | | discomfort, | |
| | | | | | aches, chills | |
| 27 | Pos- | 19.75 | 50 | f | fever, | Indiana (99%) |
| | 142 | | | | cough, | Victoria (1%) |
| | | | | | malaise, | |
| | | | | | aches | |
| 28 | Pos- | 15.83 | 53 | m | cough, sore | Indiana (67%), |
| | 143 | | | | throat, | Victoria (33%) |
| | | | | | shortness | |
| | | | | | of breath, | |
| | | | | | headache, | |
| | | | | | abdominal | |
| | | | | | pain, aches | |
| 29 | Pos- | 44.7 | 40 | m | cough, | Indiana (36%), |
| | 145 | | | | fever, | Massachusetts |
| | | | | | shortness | (27%), |
| | | | | | of breath, | Virginia (23%), |
| | | | | | chills, | Victoria (8%) |
| | | | | | headache | |
| 30 | Pos- | 23.4 | 81 | m | fever, | Indiana (100%) |
| | 146 | | | | cough, | |
| | | | | | chills | |
| 31 | Pos- | 24.42 | 30 | f | cough | Indiana (93%), |
| | 147 | | | | | Victoria (5%), |
| | | | | | | USA (1%), |
| | | | | | | Utah (0%) |
| 32 | Pos- | 18.26 | 45 | m | cough, | Indiana (100%) |
| | 149 | | | | fever, aches | |
| 33 | Pos- | 18.04 | 39 | m | fever, | Indiana (100%) |
| | 153 | | | | cough, sore | |
| | | | | | throat, | |
| | | | | | anorexia | |
| 34 | Pos- | 14.32 | 45 | m | cough, | Indiana (100%) |
| | 154 | | | | shortness | |
| | | | | | of breath, | |
| | | | | | sore throat, | |
| | | | | | headache | |
| 35 | Pos- | 17.73 | 24 | f | sore throat | Indiana (100%) |
| | 155 | | | | | |
| 36 | Pos- | 16.48 | 26 | f | cough, | Indiana (100%) |
| | 80 | | | | fever, sore | |
| | | | | | throat, | |
| | | | | | aches, | |
| | | | | | fatigue | |
| 37 | Pos- | 29.34 | 67 | f | fever, | Indiana (100%) |
| | 81 | | | | cough, | |
| | | | | | shortnes of | |
| | | | | | breath, | |
| | | | | | chills, aches | |
| 38 | Pos- | 19.4 | 41 | m | unknown | Indiana (100%) |
| | 159 | | | | | |
| 39 | Pos- | 34.5 | 37 | m | cough, | Indiana (100%) |
| | 161 | | | | fever, | |
| | | | | | weakness | |
| 40 | Pos- | 14.83 | 78 | m | repeat | Victoria (77%), |
| | 224 | | | | patient | USA (23%) |

The mean age for the 40 Indiana positive COVID-19 samples is 50 years. 30% of the Indiana positive COVID-19 patients were in the age group of 36-45 followed by 5% of the Indiana positive COVID-19 patients were in the age group 0-25. Also, 55% of the Indiana samples were from female hosts. 52.5% of the samples experienced a fever in the signs and symptoms. 62.5% of the patients experienced a cough in the signs and symptoms (Table 3).

Phylogenetic analysis of Indiana strains revealed the prevalence of mutation in Glycine spots at spike protein widespread transmission of Indiana strains At Spike Protein Codon 614, the mutation occurred which changed, D (aspartic acid) to G (glycine) was seen (Brufsky). We observed that 39 of the Indiana samples are in the G-type, while 1 Indiana sample is located in the D-type. Sequences with aspartic acid at this location are more similar to the original strain of SARS-CoV-2. Sequences with glycine at this location are more similar to the mutated strain of SARS-CoV-2. The mutation from aspartic acid to glycine seemed to create a more transmissible strain of SARS-CoV-2 in the Indiana samples. Based on previous studies and our own observations, the modification of Aspartic Acid to Glycine at Spike Protein Codon 614 is attributing to a more transmissible type of SARS-CoV-2 (Bette Korber, 2020; Brufsky; Muthukrishnan Eaaswarkhanth, 2020). Tracking the phylogenetic characteristics of SAR-CoV-2 will help with the understanding for the virus's mutational trajectory. In this study, our findings only refer to one modification, but in reality, it is probably combination of multiple mutations that cause a more transmissible and virulent strain of a virus.

Indiana SARS-CoV-2 samples suggest the prevalence of G-type At Spike Protein Codon 614, 302 of the total sample sizes had Glycine, and 148 strains had Aspartic Acid. We employed Fisher's exact test on samples with Glycine or Aspartic acid and from Indiana or not from Indiana. Our result shows a significant enrichment of Indiana samples for G-type (p-value: 1.63e-06 and the odds ratio: 21.73). i.e. a significant number of the sample size has a Glycine at Spike Protein Codon 614. In order to find sequences with the most similar mutation sites, the Nextstrain system enables the user to find which countries/provinces/states have the most similar sequences to other countries/provinces/states.

The ‘L’ type strain of SARS-CoV-2 is more abundant and transmissible than the ‘S’ type strain (Guo, 2020; Tang et al., 2020). The samples in the G (glycine) group could be defined as ‘L’ type, and the samples in the D (aspartic acid) group could be defined as ‘S’ type. Tracking mutation sites like the modification from Aspartic acid to Glycine provides insight where mutations are taking place. The phylogenetic tree shows which sequences are most similar to other sequences with similar mutations. The geographical location of the sequences plays a key role in discovering where certain locations have the same mutations

Our analysis shows that the strain starts in China, then transmits to Australia. The strain most similar to Indiana transmits from Australia to the United States. Our analysis shows that the strains appearing in Indiana with transmission lines from Australia, Michigan, Virginia, and USA. Some sequences in the data set included the division label as ‘USA’ instead of the state of origin. The transmission line that appears to be coming from Kansas is actually the representation of the USA.

The nearest branch confidence percentage for each Indiana Sample was recorded into Table 3. A Majority 65% of the Indiana Samples had a branch confidence percentage of 100% Indiana. This means most of the Indiana SARS-CoV-2 sequences are most similar to Indiana sequences included in the dataset. This is to be expected since these samples were collected in Indiana. The Indiana Sample 7 is most similar to the Michigan, US strain as seen in table 3. Some samples have variability in the branch confidence percentage. For example, Sample 29's branch confidence percentage is Indiana (36%), Massachusetts (27%), Virginia (23%), and Victoria (8%) as seen in table 3. Tracking the branches further back will show higher similarity to SARS-CoV-2 sequences from Australia.

The transmission lines from Australia appear in Michigan and Virginia before the lines appear in Indiana. This would imply that Indiana received the strain from inside the United States or directly from Australia. Tracking the transmission lines of SARS-CoV-2 would suggest the original strain of SARS-CoV-2 came from China then it was transmitted to Australia. The strain in Australia was transmitted to the United States, then the strain of SARS-CoV-2 appears in Indiana

### Materials and Methods

Sample collection: Remnant nasopharyngeal and oropharyngeal swab specimens collected from patients suspected of having COVID-19 were enrolled in this study. Patients included both outpatients and those who were admitted to the hospital for observation and treatment. Signs and symptoms displayed at the time of specimen collection included one or more of the following: fever, cough, shortness of breath, rhinitis, pharyngitis, abdominal pain, diarrhea, nausea, vomiting, and mental status change (Table 3).

Clinical investigation and diagnosis: Swab specimens were contained in viral transport medium and were tested for diagnostic purposes by either real-time reverse-transcription polymerase chain reaction (PCR) or by end-point PCR followed by bead hybridization-based detection of amplicons. Targets of the diagnostic assays included regions of the ORF1ab, N, and E genes.

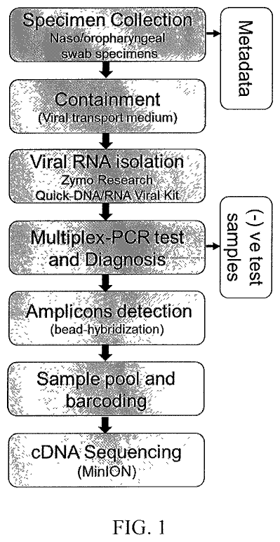

RNA isolation and sequencing: COVID-19 samples from Indiana were processed and sequenced by the MinIon Sequencer and the Artic Network as shown in FIG. 1. For this study, 40 COVID-19 positive samples were collected in viral transport media. Viral RNA was isolated using Zymo Research Quick-DNA/RNA Viral Kit (D7021) as per manufacturer's instructions. Briefly, a 25-μl reaction was set up containing 5 μl of RNA, 12.5 μl of Quantifast multiplex master mix, 0.25 μl of Quantifast RT Mix, 1 ul of Forward primer (20 uM), 1 ul of Reverse primer (20 uM) and 1 ul Probe (5 uM). Thermal cycling was performed using Qiagen Rotor-gene Q at 55° C. for 10 min for reverse transcription, followed by 95° C. for 3 min and then 45 cycles of 95° C. for 15 s, 58° C. for 30 s. ARTIC nCoV-2019 V3 primers (Ip et al.) ordered from IDT were used to amplify viral RNA into fragments of 400 bases and sequence using MinION from Oxford Nanopore Technologies (ONT).

RNA was reverse transcribed into cDNA using PCR tilling of COVID-19 from Nanopore technologies (PTC_9096_v109_revD_06Feb2020). Further, the cDNA formed was amplified using Artic nCov-2019/V3 primers. In this study, we used multiplexing and sample-pooling approach using artic primers as recommended by artic (https://artic.network/ncov-2019) and amplified the viral RNA into fragments of 400 bases. Briefly, 2.5 ul of reverse transcribed RNA was amplified using 12.5 ul Q5® Hot Start High-Fidelity 2× Master Mix (NEB, M0494), 3.7 ul of primer pool in a total reaction volume of 25 ul. Amplified products were cleaned up and end were cleaned for adapter ligation using end-repair/dA-tailing (E7546) or (cat #E7180S). Nanopore PCR barcode expansions (EXP-PBC096) were used to attach barcodes to the samples. Barcode adapters were ligated to samples using NEB Blunt/TA Ligase Master Mix (M0367). Barcodes were ligated to the sample using LongAmp Taq 2× Master Mix (e.g. NEB M0287) PCR cycles. In a PCR reaction of 15 cycles. Library preparation protocol was followed as per Nanopore Ligation Sequencing Kit (SQK-LSK109) protocol. 12 ul of end library was loaded onto the flow cell for sequencing.

For detection and quantitative estimation of SARS-CoV-2 in infected host cells, we developed an automated computational pipeline for real time data analysis and identification of the Covid19 positive samples. Our robust diagnostic pipeline can detect the viral infection in multiple samples in a single run and identify their abundance (in real time) samples obtained from human body fluids (as described previously).

Data processing: We developed a robust computational pipeline for data processing, which includes in-built base calling and demultiplexing (barcode splitting) followed by consensus building. A schematic workflow of proposed computation pipeline is shown in FIGS. 3 and 8. Briefly, the amplified and sequenced viral RNA fragments (or amplicons) were base-called and simultaneously demultiplexed using the Guppy software, installed locally. Further, the basecalled and demultiplexed barcode specific cDNA sequencing reads were processed using artic framework (Ip et al.) with default parameters (or modified, wherever applicable). We filtered the basecalled reads for each barcode using “guppyplex” module of the artic framework (Ip et al.). Next, we ran the artic “minion” module to obtain the consensus build for each barcoded sample.

Base-Calling and Demultiplexing: /path/ont-guppy/bin/guppy_basecaller -x “cuda:0” -i/path/fast5/ -s/path/basecalled/ --flowcell FLO-MIN106 --kit SQK-LSK109 --barcode_kits “EXP-PBC096” --trim_barcodes -r --nested_output_folder

*Guppy is installed locally

Data processing and consensus building: source activate artic-ncov2019. artic guppyplex--skip-quality-check --min-length 300 --max-length 700 --directory ./ nested_output_folder /BC01/--output ./merge_chopped/barcode01.fastq

artic minion --normalise 200 --threads 8 --skip-nanopolish --medaka --scheme-directory /path/artic-ncov2019/primer_schemes --read-file ./merge_chopped/barcode01.fastq nCoV-2019/V3/path/barcode01/
*artic framework is installed locally as instructed in user manual (https://artic.network/ncov-2019/ncov2019-bioinformatics-sop.html).

### Database Build

Back end: There are thousands of SARS-CoV-2 sequences on NCBI (National Center for Biotechnology Information) Virus. As of July 13^{th}, the Nextstrain build in this paper has 450 sequences. Fewer sequences were included in the analysis for better data visualization and processing. Information like collection date and location are stored in a tsv metadata file. The sequences are stored in fasta files. Metadata and sequence files are connected through the name of the strain for each SARS-CoV-2 sequence. Once the 40 consensus sequence for the Indiana samples were created, the sequences were included in a Nextstrain build. 418 Sequences from NCBI selected by the Nextstrain team were included in the analysis. The 418 NCBI sequences can be found on Nextstrain's GitHub page (Hadfield et al., 2018; Nextstrain, 2020).

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

All publications, patents and patent applications referenced herein are hereby incorporated by reference in their entirety for all purposes as if each such publication, patent or patent application had been individually indicated to be incorporated by reference.

### REFERENCES

1 Saleh F A, Sleem A. 2020. COVID-19: Test, test and test. Med Sci (Basel). 2020 Dec. 30; 9(1):E1. doi: 10.3390/medsci9010001.
2 Watzinger F, Ebner K, Lion T. 2006. Detection and monitoring of virus infections by real-time PCR. Mol Aspects Med. April-June 2006; 27(2-3):254-98. doi: 10.1016/j.mam.2005.12.001.
3 Martins-Junior R, Carney S, Goldemberg D, Bnine L, Spano L, Siquiera M, Checon R. 2014. Detection of respiratory viruses by real-time polymerase chain reaction in outpatients with acute respiratory infection. Mem Inst Oswaldo Cruz. 2014 September; 109(6):716-21. doi: 10.1590/0074-0276140046.
4 Maony J. 2008. Detection of respiratory viruses by molecular methods. Clin Microbiol Rev. 2008 October; 21(4): 716-747. doi: 10.1128/CMR.00037-07
5 Hsuang H-S, Tsai C-L, Chang J, Hsu T-C, Lin S, Lee C-C. 2017. Multiplex PCR system for the rapid diagnosis of respiratory virus infection: systematic review and meta-analysis. Clin Microbiol Infect. 2018 October; 24(10):1055-1063. doi: 10.1016/j.cmi.2017.11.018.
6 Sofi M, Hamid A, Bhat S. 2020. SARS-CoV-2: a critical review of its history, pathogenesis, transmission, diagnosis, and treatment. Biosaf. Health. 2020 December; 2(4):217-225. doi: 10.1016/j.bshea1.2020.11.002.
7 Dhama K, Khan S, Tiwara Ruchi, Sircar S, Bhat S, Malik Y, Singh K, Chaicumpa W, Bonila-Aldana K, Rodriquez-Morales, A. 2020. Coronavirus disease 2019-COVID 19. Clin. Microbiol. Rev. 2020 October; 33(4): e00028-20. doi: 10.1128/CMR.00028-02.
8 J-Q Liu, J-W Xu, C-Y Sun, J-J Wan, X-T Wang, X Chen, S L Gao. 2020. Age-stratified analysis of SARS-CoV-2 infection and case fatality rate in China, Italy, and South Korea. Eur Rev Med Pharmacol Sci. 2020 December; 24(23):12575-12578. doi: 10.26355/eurrev_202012_24054.
9 Bellan M, Patti G, Hayden E, Azzolina D, Pirisi M, Acquaviva A, Aimaretti G, . . . Sainaghi P. 2020. Fatality rate and predictors of mortality in an Italian cohort of hospitalized COVID-19 patients. Sci Rep. 2020 Nov. 26; 10(1):20731. doi: 10.1038/s41598-020-77698-44
10 Aguiar M, Stollenwerk N. 2020. Condition-specific mortality risk can explain differences in COVID-19 case fatality ratios around the globe. Public Health. 2020 November; 188:18-20. doi: 10.1016/j.puhe.2020.08.021.
11 Dhama K, Khan S, Tiwara Ruchi, Sircar S, Bhat S, Malik Y, Singh K, Chaicumpa W, Bonila-Aldana K, Rodriquez-Morales, A. 2020. Coronavirus disease 2019-COVID 19. Clin. Microbiol. Rev. 2020 October; 33(4): e00028-20. doi: 10.1128/CMR.00028-02.
12 Maiuolo J, Mollace R, Gliozzi M, Musolino V, Carresi C, Paone S, Scicchitano M, . . . Mollace V. 2020. The contribution of endothelial dysfunction in systemic injury subsequent to SARS-CoV-2 infection.
Int J Mol Sci. 2020 Dec. 6; 21(23):9309. doi: 10.3390/ijms21239309.
13 Zhang M, Zhou L, Wang J, Wang K, Wang Y, Pan X, Ma A. 2020. The nervous system—a new territory being explored of SARS-CoV-2. J Clin Neurosci. 2020 December; 82(Pt A):87-92. doi: 10.1016/j.jocn.2020.10.056.
14 Achar A, Ghosh C. 2020. COVID-19-associated neurological disorders: the potential route of CNS invasion and blood-brain relevance. Cells. 2020 Oct. 27; 9(11):2360. doi: 10.3390/cells9112360.
15 Losy J. 2020. SARS-CoV-2 infection: Symptoms of the nervous system and implications for therapy in neurological disorders. Neurol Ther. 2020 Nov. 23; 1-12. doi: 0.1007/s40120-020-00225-0.
16 Hilton J, Keeling M. 2020. Estimation of country-level basic reproductive ratios for novel coronavirus (SARS-CoV-2/COVID-19) using synthetic contact matrices. PLoS Comput. Biol. 2020 Jul. 2; 16(7):e1008031. doi: 10.1371/journal.pcbi.1008031.
17 Li Y, Wang L-W, Zhi H-P, Shen H-B. Basic reproduction number and predicted trends of coronavirus disease 2019 epidemic in the mainland of china. Infect Dis Poverty. 2020 Jul. 16; 9(1):94. doi: 10.1186/s40249-020-00704-4.
18 World Health Organization. 2021. WHO Coronavirus Disease (COVID-19) Dashboard. Retrieved from https://covid19.who.int/.
29 Kruttgen A, Cornelissen C, Dreher M, Hornef M, Imohl M, Kleines M. 2021. Comparison of the SARS-CoV-2 rapid antigen test to the real star SARS-CoV-2 PCR kit. J Virol Methods. 2021 February; 288: 114024.
20 Chaimayo C, Kaewnaphan B, Tanleing N, Athipanyasilp N, Sirijatuphat R, Chayakulkeeree M, . . . Horthongkham N. 2020. Rapid SARS-CoV-2 antigen detection assay in comparison with real-time RT-PCR assay for laboratory diagnosis of COVID-19 in Thailand. Virol J. 2020 Nov. 13; 177: 5842.
21 Scohy A, Anatharajah A, Bodeus M, Kabamba-Mukadi B, erroken A, Rodriquez-Villalobos. 2020. Low performance of rapid antigen detection test as frontline testing for COVID-19 diagnosis. J Clin Virol. 2020 August; 129:104455. doi: 10.1016/j.jcv.2020.104455.
22 Mak H, Cheng P, Lau S, Wong K, Lau C S, Lam E, . . . Tsang D. 2020. Evaluation of rapid antigen test for detection of SARS-CoV-2 virus. J Clin Virol. 2020 August; 129: 104500. doi: 10.1016/j.jcv.2020.104500
23 Deeks J, Dinnes J, Takwoingi Y, Davenport C, Spijker R, Taylor-Philips S, . . . Van den Bruel A. 2020. Antibody tests for identification of current and past infection with SARS-CoV-2. Cochrane Database Syst Rev. 2020 Jun. 25; 6(6):CD013652. doi: 10.1002/14651858.CD013652.
24 Kubina R, Dziedzic A. 2020. Molecular and serological tests for COVID-19. A comparative review of SARS-CoV-2 coronavirus laboratory and point-of-care diagnostics.
25 Alpdagtas S, Ilhan E, Uysal E, Sengor M, Ustundag C, Gunduz O. 2020. Evaluation of current diagnostic methods for COVID-19. APL Bioeng. 2020 December; 4(4): 041506. doi: 10.1063/5.0021554.
26 Osterdahl M, Lee K, Lochlainn M, Wilson S, Douthwaite S, Horsfall R, . . . Steves C. 2020. Detecting SARS-CoV-2 at point of care: preliminary data comparing loop-mediated isothermal amplification (LAMP) to polymerase chain reaction (PCR). BMC Infect Dis. 2020; 20: 783. doi: 10.1186/s12879-020-05484-8.
27 Augustine R, Hasan A, Das S, Ahmed R, Mori Y, Notomi T, . . . Thakor A. 2020. Loop-mediated isothermal amplification (LAMP): A rapid, sensitive, specific, and cost-effective point-of-care test for coronaviruses in the context of the COVID-19 pandemic. Biology (Basel). 2020 August; 9(8): 182. doi: 10.3390/biology9080182.
28 Kitagawa Y, Orihara Y, Kawamura R, Imai K, Sakai J, Tarumoto N. 2020. Evaluation of rapid diagnosis of novel coronavirus disease (COVID-19) using loop-mediated isothermal amplification. J Clin Virol. 2020 August; 129:104446. doi: 10.1016/j.jcv.2020.104446.
29 Brandsma E, Verhagen H, van de Laar T, Claas E, Cornelissen M, van den Akker E. 2020. Rapid, sensitive and specific SARS coronavirus-2 detection: a multi-center comparison between standard qRT-PCT and CRISPR based DETECTR. J Infect Dis. 2020 Oct. 10; jiaa641.doi: 10.1093/infdis/jiaa641.
30 Hou T, Zeng W, Yang M, Chen W, Ren L, Ai J, . . . Xu T. 2020. Development and evaluation of a rapid CRISPR-based diagnostic for COVID-19. PLoS Pathog. 2020 August; 16(8): e1008705.
31 Centers for Disease Control & Prevention. 2020. CDC COVID Data Tracker. Retrieved from: https://covid.cdc.gov/covid-data-tracker/.
32 United States Food & Drug Administration. 2021. Emergency use authorization. Retrieved on 14 Jan. 2020 from https://www.fda.gov/emergency-preparedness-and-response/mcm-legal-regulatory-and-policy-framework/emergency-use-authorization.
33 Centers for Disease Control & Prevention. 2020. Research use only 2019 novel coronavirus (2019-nCoV) real-time RT-PCR primers and probes. Retrieved from https://www.cdc.gov/coronavirus/2019-ncov/lab/rt-pcr-panel-primer-probes.html.
34 World Health Organization. 2020. SARS-CoV-2 PCR Protocols. Retrieved from https://www.who.int/docs/default-source/coronaviruse/whoinhouseassays/pdf
35 Tyson J, James P, Stoddart D, Sparks N, Wickenhagen A, Hall G, . . . Quick J. 2020. Improvements to the ARTIC multiplex PCR method for SARS-CoV-2 genome sequencing using nanopore.
36 http://mrprimerv.com/
37 https://www.viprbrc.org/brc/home.spg?decorator=vipr
38 https://kb.iu.edu/d/aolp
39 Untergasser A, Cutcutache I, Koressaar T, Ye J, Faircloth B, Remm M, Rozen S. 2012. Primer3—new capabilities and interfaces. Nucleic Acids Res. 2012 August; 40(15): e115. doi: 10.1093/nar/gks596.
40 https://blast.ncbi.nlm.nih.gov/Blast.cgi?PAGE_TYPE=BlastDocs&DOC_TYPE=Download
41 Curuana G, Croxatto A, Coste A, Opota O, Lamoth F, Jaton K, Grueb G. 2020. Diagnostic strategies for SARS-CoV-2 infection and interpretation of microbiological results. Clin Microbiol Infect. 2020 September; 26(9): 1178-1182. doi: 10.1016/j.cmi.2020.06.019.
42 Oliver S, Gargano J, Marin M, Wallace M, Curran K, Chamberland M, . . . Dooling K. 2020. The advisory committee on immunization practices' interim recommendation for use of Pfizer-BioNTech COVID-19 vaccine—United States, December 2020. MMWR Morb Mortal Wkly Rep. 2020 Dec. 18; 69(50):1922-1924. doi: 10.15585/mmwr.mm6950e2.
43 Baden L, Sahly H, Essink B, Kotloff K, Frey S, Novak R, . . . Zaks T. 2020. Efficacy and safety of the mrNA-1273 SARS-CoV-2 Vaccine. N Engl J Med. 2020 Dec. 30; NEJMoa2035389. doi: 10.1056/NEJMoa2035389.
44 Knoll M, Wonodi C. 2021. Oxford-AstraZeneca COVID-19 vaccine efficacy. Lancet. 2021 Jan. 9; 397(10269):72-74. doi: 10.1016/S0140-6736(20)32623-4.
45 Lee S, Lee D H. 2020. Lessons learned from battling COVID-19: the Korean experience. Int J Environ Res Public Health. 2020 October; 17(20): 7548. doi:10.3390/ijerph17207548.
46 Wells C, Townsend J, Pandey A, Moghadas S, Krieger G, Singer B, . . . Galvani A. 2021. Optimal COVID-19 quarantine and testing strategies. Nature 12; 356 (2021): 2450.
47 Kober B, Fischer W, Gnanakaran S, Yoon H, Theiler J, Abfalterer W, . . . Montefiori D. Tracking changes in SARS-CoV-2 spike: Evidence that D614G increases infectivity of the COVID-19 virus. Cell. 2020 Aug. 20; 182(4): 812-827.e19. doi: 10.1016/j.cell.2020.06.043.

### ADDITIONAL REFERENCES & LITERATURE

Simmonds P, Aiewsakun P. Virus classification—where do you draw the line? Arch Virol. 2018; 163(8):2037-46. Epub 2018/07/25. doi: 10.1007/s00705-018-3938-z. PubMed PMID: 30039318; PMCID: PMC6096723.
Saingam P, Li B, Yan T. Use of amplicon sequencing to improve sensitivity in PCR-based detection of microbial pathogen in environmental samples. J Microbiol Methods. 2018; 149:73-9. Epub 2018/05/11. doi: 10.1016/j.mimet.2018.05.005. PubMed PMID: 29746923.
Dundas N, Leos N K, Mitui M, Revell P, Rogers B B. Comparison of automated nucleic acid extraction methods with manual extraction. J Mol Diagn. 2008; 10(4):311-6. Epub 2008/06/17. doi: 10.2353/jmoldx.2008.070149. PubMed PMID: 18556770; PMCID: PMC2438199.
Kok T, Wati S, Bayly B, Devonshire-Gill D, Higgins G. Comparison of six nucleic acid extraction methods for detection of viral DNA or RNA sequences in four different non-serum specimen types. J Clin Virol. 2000; 16(1):59-63. Epub 2000/02/19. doi: 10.1016/s1386-6532(99)00066-9. PubMed PMID: 10680742.
Miller S, Seet H, Khan Y, Wright C, Nadarajah R. Comparison of QIAGEN automated nucleic acid extraction methods for CMV quantitative PCR testing. Am J Clin Pathol. 2010; 133(4):558-63. Epub 2010/03/17. doi: 10.1309/AJCPE5VZL1ONZHFJ. PubMed PMID: 20231608.
Rasmussen T B, Uttenthal A, Hakhverdyan M, Belak S, Wakeley P R, Reid S M, Ebert K, King D P. Evaluation of automated nucleic acid extraction methods for virus detection in a multicenter comparative trial. J Virol Methods. 2009; 155(1):87-90. Epub 2008/10/28. doi: 10.1016/j.jviromet.2008.09.021. PubMed PMID: 18952126.
Lewandowski K, Bell A, Miles R, Came S, Wooldridge D, Manso C, Hennessy N, Bailey D, Pullan S T, Gharbia S, Vipond R. The Effect of Nucleic Acid Extraction Platforms and Sample Storage on the Integrity of Viral RNA for Use in Whole Genome Sequencing. J Mol Diagn. 2017; 19(2):303-12. Epub 2017/01/04. doi: 10.1016/j.jmoldx.2016.10.005. PubMed PMID: 28041870.
Verheyen J, Kaiser R, Bozic M, Timmen-Wego M, Maier B K, Kessler H H. Extraction of viral nucleic acids: comparison of five automated nucleic acid extraction platforms. J Clin Virol. 2012; 54(3):255-9. Epub 2012/04/17. doi: 10.1016/j.jcv.2012.03.008. PubMed PMID: 22503856.
Midha M K, Wu M, Chiu K P. Long-read sequencing in deciphering human genetics to a greater depth. Hum Genet. 2019; 138(11-12):1201-15. Epub 2019/09/21. doi: 10.1007/s00439-019-02064-y. PubMed PMID: 31538236.
Depledge D P, Wilson A C. Using Direct RNA Nanopore Sequencing to Deconvolute Viral Transcriptomes. Curr Protoc Microbiol. 2020; 57(1):e99. Epub 2020/04/08. doi: 10.1002/cpmc.99. PubMed PMID: 32255550.
Viehweger A, Krautwurst S, Lamkiewicz K, Madhugiri R, Ziebuhr J, Holzer M, Marz M. Direct RNA nanopore sequencing of full-length coronavirus genomes provides novel insights into structural variants and enables modification analysis. Genome research. 2019; 29(9):1545-54. Epub 2019/08/24. doi: 10.1101/gr.247064.118. PubMed PMID: 31439691; PMCID: PMC6724671.
Depledge D P, Srinivas K P, Sadaoka T, Bready D, Mori Y, Placantonakis D G, Mohr I, Wilson A C. Direct RNA sequencing on nanopore arrays redefines the transcriptional complexity of a viral pathogen. Nat Commun. 2019; 10(1):754. Epub 2019/02/16. doi: 10.1038/s41467-019-08734-9. PubMed PMID: 30765700; PMCID: PMC6376126.
Ji P, Aw T G, Van Bonn W, Rose J B. Evaluation of a portable nanopore-based sequencer for detection of viruses in water. J Virol Methods. 2020; 278:113805. Epub 2020/01/01. doi: 10.1016/j.jviromet.2019.113805. PubMed PMID: 31891731.
Tweed J A, Gu Z, Xu H, Zhang G, Noun P, Li M, Steenwyk R. Automated sample preparation for regulated bioanalysis: an integrated multiple assay extraction platform using robotic liquid handling. Bioanalysis. 2010; 2(6):1023-40. Epub 2010/11/19. doi: 10.4155/bio.10.55. PubMed PMID: 21083206.
Koressaar T, Remm M. Enhancements and modifications of primer design program Primer3. Bioinformatics. 2007; 23(10):1289-91. Epub 2007/03/24. doi: 10.1093/bioinformatics/btm091. PubMed PMID: 17379693.
Kim H, Kang N, An K, Kim D, Koo J, Kim M S. MRPrimerV: a database of PCR primers for RNA virus detection. Nucleic Acids Res. 2017; 45(D1):D475-D81. Epub 2016/12/03. doi: 10.1093/nar/gkw1095. PubMed PMID: 27899620; PMCID: PMC5210568.
Tham C Y, Tirado-Magallanes R, Goh Y, Fullwood M J, Koh B T H, Wang W, Ng C H, Chng W J, Thiery A, Tenen D G, Benoukraf T. NanoVar: accurate characterization of patients' genomic structural variants using low-depth nanopore sequencing. Genome biology. 2020; 21(1):56. Epub 2020/03/05. doi: 10.1186/s13059-020-01968-7. PubMed PMID: 32127024; PMCID: PMC7055087.
Hadfield J, Megill C, Bell S M, Huddleston J, Potter B, Callender C, Sagulenko P, Bedford T, Neher R A. Nextstrain: real-time tracking of pathogen evolution. Bioinformatics. 2018; 34(23):4121-3. Epub 2018/05/24. doi: 10.1093/bioinformatics/bty407. PubMed PMID: 29790939; PMCID: PMC6247931.
Stano M, Beke G, Klucar L. viruSITE-integrated database for viral genomics. Database: the journal of biological databases and curation. 2016; 2016. Epub 2016/12/28. doi: 10.1093/database/baw162. PubMed PMID: 28025349; PMCID: PMC5199161.
Brister J R, Ako-Adjei D, Bao Y, Blinkova O. NCBI viral genomes resource. Nucleic Acids Res. 2015; 43(Database issue):D571-7. Epub 2014/11/28. doi: 10.1093/nar/gku1207. PubMed PMID: 25428358; PMCID: PMC4383986.

What is claimed:

1. A method to characterize at least one virus in at least one human patient, the method comprising:
(a) extracting a viral polynucleotide from a biological sample from the at least one human patient,
(b) sequencing the viral polynucleotide to generate viral polynucleotide sequence data; and,
(c) characterizing the viral polynucleotide sequence data.

2. A method according to claim 1, where the step of sequencing the viral polynucleotide is performed to generate either targeted viral polynucleotide sequence data or single molecule viral genome data.

3. A method according to claim 1, where the step of characterizing viral polynucleotide sequence data is performed to reconstruct the genome of the virus, to determine evolutionary relationships and abundance of the viral specie, and/or to determine a clinical risk associated with the presence of the virus in the patient.

4. A method according to claim 1, where the method is a point-of-care, real-time method to characterize the at least one virus from a plurality of different biological samples from human patients

5. A method according to claim 1, where the viral polynucleotide is a viral RNA or DNA

6. A method according to claim 1, where the at least one virus is at least two viruses and one virus is a coronavirus

7. A method according to claim 4, where the coronavirus is severe acute respiratory syndrome coronavirus 2 (SARS-CoV-2)

8. A method according to claim 1, where the biological sample from the at least one human patient is a nasopharyngeal sample, a mucus sample, a saliva sample, a sputum sample, a bronchial aspirate and a serum sample.

9. A method according to claim 1, further comprising the step of processing the viral polynucleotide to add or to remove a unique barcode identifier with the viral polynucleotide where the barcode identifier represents metadata identifying a source sample from which the biological sample was taken and the unique barcode identifier is configured to form a unique, repeatable, characteristic signature when read during the sequencing step.

10. A method according to claim 1, where the sequencing step is a high-throughput sequencing step.

11. A method according to claim 10, where the sequencing step is performed by a nanopore process and the nanopore process utilizes an Oxford Nanopore MinION sequencer.

12. A method according to claim 1, where the step of characterizing the targeted viral polynucleotide sequence data includes detecting whether a virus is present in the biological sample.

13. A method according to claim 1, where the step of characterizing the targeted viral polynucleotide sequence data includes providing strain information about a virus that is present in the biological sample.

14. A method according to claim 1, where the step of characterizing the targeted viral polynucleotide sequence data includes providing viral burden information about a virus that is present in the biological sample.

15. A method according to claim 1, where the step of characterizing the targeted viral polynucleotide sequence data is completed upon obtaining a desired result.

16. A method according to claim 1, where the sequencer generating the targeted viral polynucleotide sequence data is stopped, upon determining the presence of the virus in a sample in real time.

17. A method according to claim 1, where the sequenced viral genomes from an individual patient sample provide the identity of the strain, species and abundance of the viruses enabling real time understanding of the evolution of the virus.

18. A method according to claim 1, where the sequencing data yields information on co-infection of multiple viruses in a patient sample to facilitate therapeutic decisions and combinatorial vaccine therapies.

19. A method according to claim 1, where the data analysis of the resulting sequencing data can be performed locally or on a remote server to provide information to the end user on smart phone or mobile devices.

20. A method according to claim 1, where the experimental protocol for isolating the virus can involve the use of specific primers targeting one or more virus of interest from a multitude of viruses in a biological sample.

21. A method according to claim 1, where the experimental protocol for isolating the virus can involve sequencing one or more virus species of interest without the use of primers by directly sequencing the RNA species in a biological sample without any amplification step.
